(19) United States
(12) Patent Application Publication
(10) Pub. No.: US 2026/0355803 A1
(43) Pub. Date: Nov. 12, 2026

(54) METHOD AND APPARATUS FOR PRODUCING A HOLLOW CYLINDER FOR A MEDICAL APPLICATION

(71) Applicant: BIOTRONIK AG, Buelach (CH)

(72) Inventor: Alexander Heuss, Insel Poel (DE)

(21) Appl. No.: 19/122,402

(22) PCT Filed: Nov. 7, 2023
(86) PCT No.: PCT/EP2023/081007

§ 371 Date: Apr. 18, 2025

(30) Foreign Application Priority Data
Dec. 8, 2022 (EP) 22212146.9

(51) Int. Cl. B21C 37/20; A61F 2/04; B21C 23/21; B21C 31/00

(52) U.S. Cl. CPC B21C 37/20; A61F 2/04; B21C 23/21; B21C 31/00; A61F 2240/001

(57) ABSTRACT

An apparatus for producing a hollow cylinder for an implant. A recipient includes a recipient chamber configured to receive a blank. A punch is configured to be guided in the recipient chamber. Aa mandrel is configured to form an inner diameter of the hollow cylinder. A die is configured to form an outer diameter of the hollow cylinder. The die is supported by at least one supporting element positioned between the recipient and the die. The supporting element is moveable in a way to enable contact between the die and the punch and/or contact between die and recipient.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP 2023/081007, which was filed Nov. 7, 2023, which application claimed priority from EP Application 22212146.9, which was filed Dec. 8, 2022.

### FIELD OF THE INVENTION

A field of the invention concerns devices for manufacturing medical implants including a hollow cylinder.

### BACKGROUND

Workpieces or semi-finished products in the form of hollow cylinders are used in many areas of medical technology. Stents for keeping hollow orifices or vessels open are an important example. Stents have a body in the form of a tubular or hollow-cylindrical basic grid which is open at both longitudinal ends. Often, hollow-cylindrical semi-finished products are used as the initial shape for such a body, from which the basic grid is then cut out, for example by a laser.

Stents are used in blood vessels, especially in the coronary arteries, to prevent them from being blocked again after they have been dilated; this type of treatment is known as stent angioplasty. On the other hand, stents are used in cancer treatment to keep open constrictions of the airways (trachea), bile ducts or esophagus caused by malignant tumors. In addition to the primary goal of preventing a new vessel occlusion, stents can also fix vascular deposits and improve the blood flow in the vessel, as they smoothen the surface of the vessel interior by being pressed against the vessel wall.

In principle, the required workpieces or semi-finished products in the form of hollow cylinders can be produced by forming processes. One possible method is extrusion or impact extrusion, which is a well-known manufacturing process for thin-walled tubes. In this process, a drilled blank is placed on a mandrel of a punch, which acts as an inner tool and thus defines the final inner diameter of the hollow cylinder, and is then pressed through a die, acting as the outer tool for defining the cylinder's outer diameter. Comparatively high degrees of forming can be achieved in a single process step, allowing to realize good mechanical properties, i.e., small grains, high strength and moderate elongation at break.

An indirect variant of extrusion presses a punch together with the die into the recipient by the die and/or the die is moved towards the recipient and or/the recipient is also moved. This process produces less frictional force, as there is less or no relative movement between the punch and the recipient. The efficiency of the pressing movement is therefore higher than in the direct extrusion process.

However, the dimensions of a medical product, particularly of a stent, are often in the range of millimeters, which limits the length of the die due to resulting bends or even fractures. The formation of bends and fractures can be approximately predicted by the third case of Euler's buckling formula.

### SUMMARY OF THE INVENTION

An apparatus for producing a hollow cylinder for an implant. A recipient includes a recipient chamber configured to receive a blank. A punch is configured to be guided in the recipient chamber. Aa mandrel is configured to form an inner diameter of the hollow cylinder. A die is configured to form an outer diameter of the hollow cylinder. The die is supported by at least one supporting element positioned between the recipient and the die. The supporting element is moveable in a way to enable contact between the die and the punch and/or contact between die and recipient.

A process for producing a hollow cylinder for an implant extrudes a blank that is at least partially inserted into a recipient chamber of a recipient by pressing a punch and a die both into the recipient. A mandrel is driven into the blank to create a hollow cylinder in the blank with an inside diameter being defined by the mandrel and an outside diameter being defined by the die. At least one supporting element positioned between the recipient and the die is moved in a way to enable contact between the die and the punch and/or contact between die and recipient.

### BRIEF DESCRIPTION OF THE DRAWINGS

In the drawings:

FIG. 1 a schematic view of an apparatus according to the invention,

FIG. 2 a and b schematic view of a first embodiment of the supporting element and

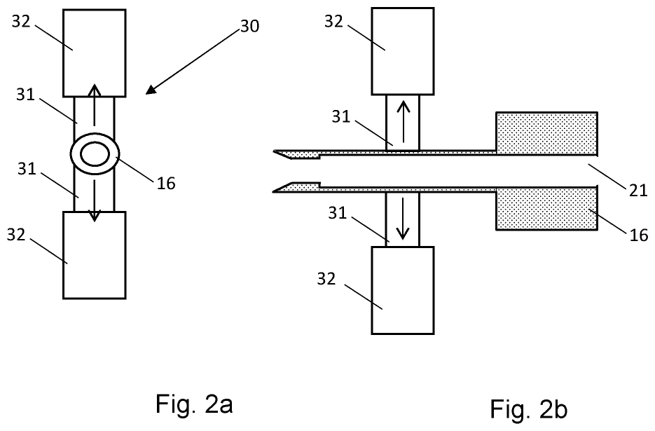

FIG. 3 a and b schematic view of a second embodiment of the supporting element

### DETAILED DESCRIPTION OF THE PREFERRED EMBODIMENTS

A preferred apparatus features a recipient with a recipient chamber which can receive a blank. Further, the apparatus includes a punch featuring a press disc and a mandrel, both configured to be also inserted at least partly into the recipient chamber. Moreover, the extrusion device includes a die, also called matrix. The die is supported by at least one supporting element positioned between the recipient and the die. The punch and the die are arranged in such a way the die is pressed into the recipient so that a hollow cylinder is formed. The movement can be made by the recipient and/or the die.

Simultaneously the mandrel is also pressed through, having a length greater than the length of the resulting hollow cylinder. In a first variant, the mandrel can be guided through the punch and driven into the blank independently of its movement in such a way that it penetrates it completely. In a second variant, the mandrel is attached to the punch and already penetrates the blank completely during insertion, which requires a tubular design of the blank, so a complete opening is necessary. In both variants, a hollow cylinder is built with an inner diameter formed by the mandrel and an outer diameter formed by the die.

Depending on the used mandrel, a drill hole is optionally foreseen in the blank for a more accurate positioning of the mandrel. The drill hole can be designed as a blind hole or as a complete opening, depending on the used arrangement of the mandrel. It is possible to first join the blank and the punch by the mandrel and then insert them into the recipient chamber of the recipient or to first insert the blank into the recipient chamber and then insert the mandrel and the punch.

The supporting element is moveable in such a way that it can be removed from the pressing direction such that contact between the die and the punch and/or contact between die and recipient is made possible. This enables a movement until the die has arrived at the front surface of the punch and has thus pressed the entire blank as the die has the length of the blank and the diameter of the inner diameter of the recipient chamber.

So, also very thin and/or long dies can be used and, therefore, also very thin and/or long hollow cylinders can be produced.

As a preferred embodiment the apparatus includes at least two, preferably three, support elements. These support elements are arranged in series along the direction of the pressing direction and thus support the die at different positions. This enables support at different points and thus the use of particularly long dies.

Alternatively or additionally the apparatus may include at least two, preferably three, support elements which are arranged at one position, in particular arranged circumferentially at the one position in a plane perpendicular to the pressing direction and thus support the die with several bearing surfaces. By supporting the die from several directions, the support element reliably prevents not only gravity-induced downwards bending, but also bending in other directions. The use of two support elements is the simplest variant, because the number of elements used is minimized. If, on the other hand, three elements or more are used, this has the advantage of creating a statically determined system.

Moreover, the at least one supporting element is movable towards the die or the recipient. so that the supporting effect is maintained for as long as possible. This can be achieved, for example, by mounting the supporting element itself on rollers or a sledge and thus, being displaced by the matrix or the recipient in pression direction when touched.

In a preferred embodiment, the supporting element is designed as a lunette, also called steady rest which is formed as a ring around the die, which is, therefore stabilized particularly well.

The at least one support element preferably has at least one at least partially convexly or concavely formed bearing surface. Having a concave form, the supporting element can therefore fit very well, especially on round dies, by taking up their outer diameter and thus creating a larger contact surface. On the opposite, a convex formed bearing surface facilitates the sliding of the die by minimizing the contact surface, thus promoting very uniform pressing and, as a consequence, a very homogeneous product.

Moreover, the at least one support element is hydraulically, pneumatically or electrically displaceable. Any type of drive can be used, but it is best to use the already available means, such as existing compressed air connections for a pneumatic drive.

As an alternative to a method with a drive, which enables a movement preferably essentially horizontal to the pressing direction, the at least one supporting element can also be rotatable or tiltable or can be folded away via a torsion spring out of the direction of movement on contact. This allows very easy movement without requiring an additional drive.

In this context, it is particularly preferred that a wiper is provided on the recipient or on the die for the at least one support element. This wiper displaces the rotatable or tiltable support element out of the direction of movement on contact. Thereby, the at least one support element can be removed without additionally requiring a switch and a drive, which makes the design easier and more interference resistant.

In another preferred embodiment, the apparatus also includes two rollers provided downstream of the die, wherein the rollers define a roll gap through which the hollow cylinder is guided. Thereby, a supporting tension can be applied to the material in the described manner, which improves the mechanical properties of the hollow cylinder.

This apparatus is advantageously used to carry out a process for producing a hollow cylinder for a medical application or for a medical product, in particular for an implant. All features of the process described should also apply to their formation in the apparatus according to the invention and vice versa.

During the pressing process, the blank is moved into the recipient with the punch. From the other side, the die presses on the blank, forcing it through the opening of the die. The movement continues until the die has reached the front surface of the punch and has thus pressed the entire blank. This means that the die must have the length of the blank and the diameter of the inner diameter of the recipient chamber. Additionally, a mandrel is foreseen to simultaneously form an inner diameter of a resulting hollow cylinder.

The thinner the blank and thus the inner diameter of the recipient, the thinner the die and the smaller the volume of the blank and thus the length of the semi-finished product to be produced. If the length of the blank is increased in order to produce longer semi-finished products, the die length must also be increased. This, however, increases the risk of buckling.

Therefore, at least one supporting element is positioned between the recipient and the die, and that the supporting element is moved in a way that contact between the die and the punch and/or contact between die and recipient is made possible.

Preferably, the blank is heated together with the punch in the recipient to a temperature in the range of 200 to 300 °C, in particular cases in a range between 250 and 280 °C. This enables a more homogeneous forming with regard to the grain size distribution.

In other preferred embodiments, the movement of the at least one support element is triggered by an electrical signal or a mechanical switch. While a mechanical switch is often used and easier to construct, an electrical signal generally has the advantage of greater accuracy. In addition, the signal can be flexibly adjusted depending on the matrices used.

The advantages of this invention can be summarized as follows: It is possible to use long, thin starting materials (blanks). Independent from the specific length, the risk of buckling is reduced and, thereby, reduction of tool costs is achieved.

Moreover, the reduced risk of buckling enables also lower forming temperatures as lower temperatures cause an increase in press force. This in turn increases the risk of buckling which is no reduced by the supporting element(s). Concluding, less energy is required. So tools can be used that are less heat resistant, whereby tool costs are reduced further.

As already described, the use of stents produced with such a method can widen constricted areas in the vessels. However, such an extraneously body in the body carries the risk of a gradual ingrowth of the stent, which can lead to a de novo vessel occlusion in the worst case.

One approach to solve this problem is to make implants and especially stents from a biodegradable material. Biodegradation is understood to be the result of hydrolytic, enzymatic and other metabolic degradation processes in the living organism, which are mainly caused by the body fluids coming into contact with the biodegradable material of the implant, and lead to a gradual dissolution of the structures of the implant including the biodegradable material. The implant loses its mechanical integrity at a certain point due to this process. The term biocorrosion is often used synonymously with the term biodegradation. The term bioresorption additionally includes the subsequent resorption of the degradation products by the living organism.

Materials suitable for the body of biodegradable implants may contain, for example, polymers or metals. Biodegradable magnesium implants, in particular magnesium stents, have proven to be particularly promising for the above-mentioned target corridor of degradation.

In addition to any semi-finished product or hollow cylinder obtained by the process as according to the invention, the present invention particularly protects the implementation of the process using a magnesium alloy and a hollow cylinder made of a magnesium alloy.

Further objectives, features, advantages and possible applications of the invention can also be taken from the following description of the attached figures and the example. All features described and/or illustrated form the subject matter of the invention per se or in any combination, independent of their inclusion in the individual claims or their back-references.

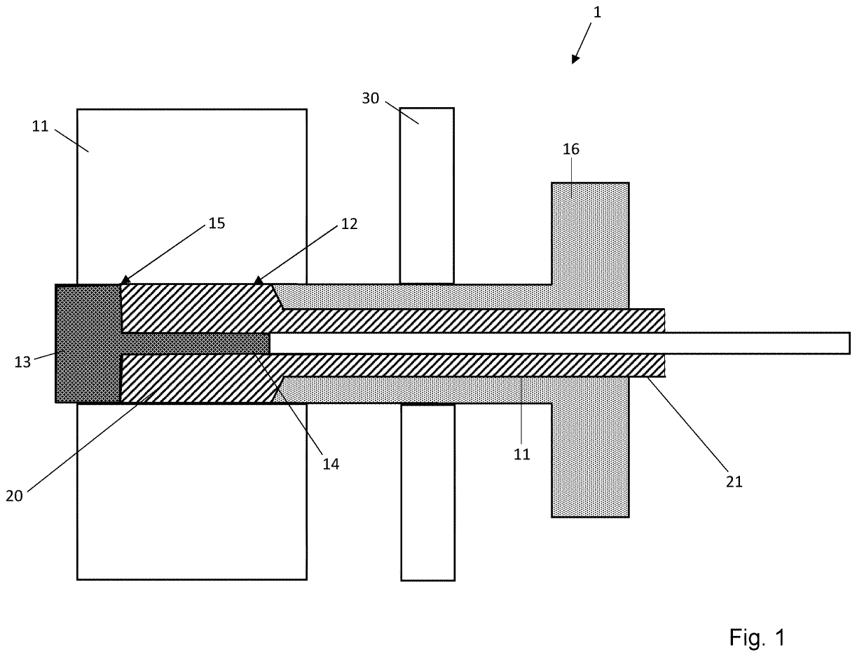

FIG. 1 shows an apparatus 1 according to the invention. This apparatus features a recipient 11, a punch 13 and a die 16.

A recipient chamber 12 is provided in the recipient 11. The recipient chamber 12 is designed in such a way that a punch 13 and a blank 20 can be inserted at least partially. The punch 13 is designed in such a way that it has a press disc 15 on one side with which it can press on blank 20. Moreover, a mandrel 14 is provided on press disc 15, which can at least partially penetrate the blank 20.

During the pressing process, the die 16 and the punch 13 are moved relative to each other, either by moving the punch with the press disc 15 towards the die 16 or by moving the die 16 in the opposite direction into the recipient chamber 12. This deforms the blank 20 in such a way that its outer diameter assumes the round shape defined by the die 16 and, at the same time, the mandrel 14 penetrates its interior in such a way that a cylindrical cavity is created. The forming thus produces a hollow cylinder 21, featuring an outer diameter determined by the die 16 and an inner diameter determined by the mandrel 14.

In this extrusion press device, at least one supporting element 30 is foreseen, which braces the die 16 to avoid bending or even breaking. This supporting element 30 is movable so that it does not partially block the path when the die and punch are moving towards each other and can further be removed for a certain shorter unsupported movement of the die to such an extent that die and punch or die and recipient can touch each other.

The front view 2a shows that two supporting elements 30 are provided, which engage with the die 16 offset by 180° and thus stabilize it particularly efficiently. The supporting elements 30 are also divided into two areas, whereby a moveable area 31 is designed in such a way that it can be moved mechanically, pneumatically, hydraulically, magnetically or electrically into and out of a static area 32.

From the side view 2b it can be seen that after moving these areas 31 into static areas 32, the die 16 can pass the supporting elements 30. In this embodiment, the movement is particularly advantageous in that it is essentially orthogonal to the direction of movement of the device and/or the recipient, and thus, the distance to be moved is minimized.

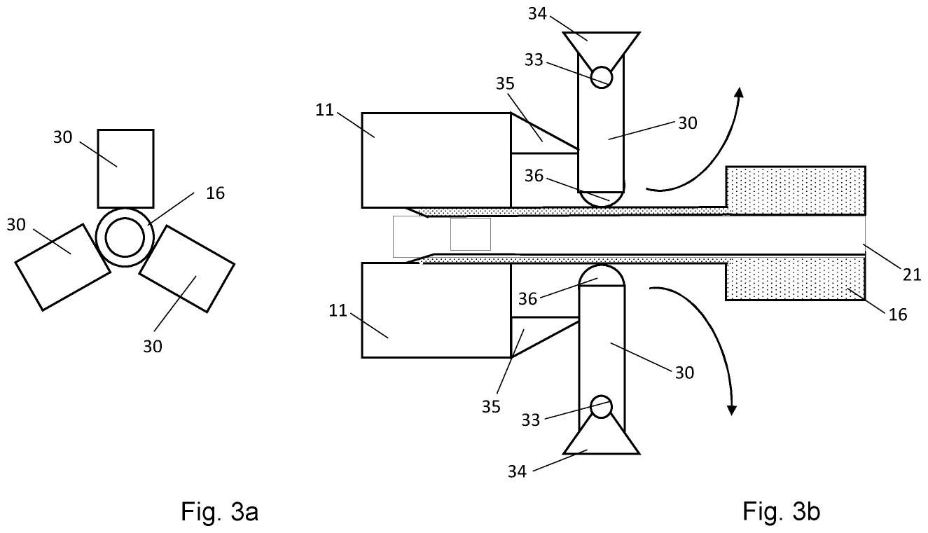

FIG. 3a shows in front view an embodiment according to the invention with three supporting elements 30 encompassing the die 16.

In the side view 3b it is shown that the supporting elements 30 are each fixed with a rotatable mounting 33 and a holder 34. When the recipient 11 moves, the wipers 35 press against the supporting elements 30 so that they can be displaced due to the rotatable mounting 33 and do not block the direction of movement between the recipient 11 and the die 16. In the same way, such wipers 35 can also be provided on the die 16.

In addition, in this view, a convex design of the surfaces 36 of the supporting elements 30 can also be seen, which prevents a blocking of the die 16 and supporting elements 30. This is particularly important in this pivotable embodiment.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

### LIST OF REFERENCE NUMBERS

1 apparatus according to the invention
10 extrusion press device
11 recipient
12 recipient chamber
13 punch
14 mandrel
15 press disc
16 die
20 blank
21 hollow cylinder
30 supporting element(s)
31 moveable area
32 static area
33 rotatable mounting
34 holder
35 wiper(s)
36 surface of the supporting element

1. An apparatus for producing a hollow cylinder for an implant, comprising:
a recipient comprising a recipient chamber configured to receive a blank;
a punch configured to be guided in the recipient chamber;
a mandrel configured to form an inner diameter of the hollow cylinder; and
a die configured to form an outer diameter of the hollow cylinder, wherein the die is supported by at least one supporting element positioned between the recipient and the die, the supporting element being moveable in a way to enable contact between the die and the punch and/or contact between die and recipient.

2. The apparatus according to claim 1, comprising at least two, supporting elements arranged in series to support the die at different positions.

3. The apparatus according to claim 1, comprising three supporting elements circumferentially arranged at one position in a plane perpendicular to a pressing direction to support the die from different directions.

4. The apparatus according to claim 1, wherein the at least one supporting element is movable towards the die or the recipient.

5. The apparatus according to claim 1, wherein the at least one supporting element comprises a lunette.

6. The apparatus according to claim 1, wherein the at least one supporting element comprises at least one at least partially convexly or concavely formed bearing surface.

7. The apparatus according to claim 1, wherein the at least one supporting element is mechanically, hydraulically, pneumatically magnetically or electrically displaceable.

8. The apparatus according to claim 1, wherein the at least one supporting element is rotatable or tiltable.

9. The apparatus according to claim 1, comprising a torsion spring arranged to permit the at least one canto be folded away.

10. The apparatus according to claim 8, comprising a wiper on the recipient or on the die. wherein the wiper is arranged to displace the at least one supporting element out of the direction of movement between the recipient and the die.

11. The apparatus according to claim 1 the apparatus further comprising two rollers provided downstream of the die, the rollers being arranged to delimit a rolling gap through which the hollow cylinder is guided.

12. A process for producing a hollow cylinder for an implant, comprising:
extruding a blank that is at least partially inserted into a recipient chamber of a recipient by pressing a punch and a die both into the recipient;
driving a mandrel into the blank to create a hollow cylinder in the blank with an inside diameter being defined by the mandrel and an outside diameter being defined by the die,
moving at least one supporting element positioned between the recipient and the die in a way to enable contact between the die and the punch and/or contact between die and recipient.

13. The process according to claim 11, comprising triggering movement of the at least one supporting element by an electrical signal.

14. The process according to claim 11, comprising triggering movement of the at least one supporting element by a mechanical switch.

15. (canceled)
